(19) United States
(12) Patent Application Publication
(10) Pub. No.: US 2012/0109194 A1
(43) Pub. Date: May 3, 2012

(54) Suspensory graft fixation with adjustable loop length

(75) Inventors: Peter C. Miller, Largo, FL (US); Steven E. Fitts, Largo, FL (US); Giuseppe Lombardo, New Port Richey, FL (US)

(73) Assignee: LINVATEC CORPORATION

(21) Appl. No.: 13/317,055

(22) Filed: Oct. 7, 2011

Related U.S. Application Data
(60) Provisional application No. 61/455,897, filed on Oct. 28, 2010.

(51) Int. Cl. A61B 17/04

(52) U.S. Cl. 606/232

(57) ABSTRACT

A suspensory graft ligament fixation device is shown to be particularly suitable for maximizing the contact between a soft tissue graft and the bone tunnel prepared to receive the graft. The suspensory fixation device has an elongated anchor member adapted to be transversely situated at the exit of the bone tunnel. A loop member is suspended transversely from the anchor member and has a loop length which is adjustable. When a graft ligament is attached to the saddle end of the loop, the length of the loop member may be shortened to pull the graft member into the bone tunnel until it bottoms out at the floor of the bone tunnel.

This application claims the benefit of pending prior U.S. Provisional Patent Application Ser. No. 61/455,897, filed Oct. 28, 2010 by Peter C. Miller et al. for Adjustable Loop for Short Tunnel ACL.

### BACKGROUND OF THE INVENTION

1. Field of the Invention

This invention relates to surgical devices for the repair and reconstruction of soft tissue injuries. In particular, this invention relates to devices and methods for the surgical implantation of artificial ligament grafts. Still more particularly, the invention relates to the surgical repair of torn anterior cruciate ligament grafts.

2. Description of the Prior Art

The repair and reconstruction of torn or damaged soft tissues is a common surgical procedure. For example, replacement graft ligaments may be secured at the site of the original ligament. The procedure generally involves drilling bone tunnels into adjacent bones at the site of the original ligament and securing a graft ligament within these bone tunnels. In many applications, such as in the knee joint, such procedures may be performed arthroscopically. The graft ligament may be an autograft, an allograft, a xenograft, and/or it may be totally artificial and synthetic. The most common types of anterior cruciate ligament (ACL) grafts, for example, include ones which may be bone-patellar-tendon-bone or soft tissue (such as semitendinosus and gracilis tendons), both types harvested by techniques well known to those skilled in the art.

The graft ligaments are secured within the bone tunnels in a variety of ways. Of prime importance is the degree to which they can withstand pullout forces prior to complete healing. For example, it is known to use interference screws inserted parallel to the tunnel axis to compress the ends of the graft ligament against the walls of the bone tunnel to secure the graft ligament and promote tissue in-growth.

Suspensory graft fixation devices have been developed to secure a graft ligament in a bone tunnel. One such device is described in U.S. patent application Ser. No. 11/804,195, filed May 17, 2007, entitled Graft Fixation Implant, assigned to the assignee hereof and incorporated by reference herein. Suspensory graft fixation devices work with a bone tunnel and generally take the form of an elongated member having an axis and a pair of suture receiving apertures symmetrically situated on the axis on opposite sides of the longitudinal center of the elongated member. In ACL procedures the elongated member, often called a button, is adapted to be situated transversely to the exit opening of the bone tunnel on the lateral cortex so that a suture loop suspended from the button can extend into the bone tunnel from the suture receiving apertures and support one end of a graft ligament passed through the loop. In arthroscopic procedures such as ACL reconstruction the elongated member supports a graft ligament and is rotated into alignment with the previously formed bone tunnel in order to enable it to be inserted into the proximal opening of the bone tunnel and to exit at the distal end on the lateral femur. For such suspensory graft fixation devices to be able to support a graft ligament and to be properly transversely situated at the exit of the bone tunnel, the suture loop and the bone tunnel must both be long enough to enable the elongated member to “flip” from an axially aligned orientation to a transverse orientation when it exits the bone tunnel.

Since the supporting loop of such a suspensory device is most often of a fixed length, graft fixation requires preparation of a graft ligament of predetermined length. Furthermore, because prior art suspensory graft fixation devices have fixed loop lengths they are produced in multiple sizes (ranging, for example, from loop lengths of 15 mm to 60 mm in 5 mm increments in the case of XO Button™ implants made by ConMed Linvatec, Largo, Fla.) in order to accommodate various graft and tunnel lengths that may be encountered during a surgical procedure. The fixed graft length and variation in tunnel and loop lengths makes prior art suspensory ligament fixation challenging.

Recently, suspensory devices have been made with adjustable loop lengths as taught by U.S. patent application 2010/0256677, (Albertorio et al.) published Oct. 7, 2010 and entitled Integrated Adjustable Button-Suture-Graft Construct with Two Fixation Devices. It has been found that the adjustability of the loop length of a suspensory graft fixation device may be achieved in a manner considerably less complex than that described in the aforementioned publication.

At times surgeons may encounter situations where they cannot produce a bone tunnel of adequate length to receive a ligament graft suitable for suspensory fixation. A predetermined length of graft ligament is required to engage the bone tunnel for proper healing. For example, a so-called short tunnel ACL reconstruction may present a relatively small (narrow) femur which does not enable formation of an adequately long bone tunnel which means, in turn, the suspensory anchor member cannot be advanced far enough out of the tunnel to flip. Use of an adjustable loop in such situations could nevertheless enable the surgeon to proceed with a suspensory-type repair. Accordingly it is an object of this invention to produce a suspensory graft ligament repair system suitable for short tunnel repairs.

### SUMMARY OF THE INVENTION

These and other objects of this invention are achieved by a suspensory graft fixation device for securing a ligament graft in a bone tunnel comprising an elongated anchor member adapted to be situated transversely adjacent the exit of the bone tunnel, a graft supporting element adapted to support the ligament graft a predetermined variable distance away from the exit, and a pulley-type means for adjusting the distance.

In one aspect of the invention the graft supporting element comprises a plurality of loops formed from a single wound strand of filamentous material having two ends movable relative to each other.

Another aspect of the invention is the method comprising the steps of providing a suspensory graft fixation device as described above and pulling at least one end of the filamentous material in order to shorten the loop length.

### BRIEF DESCRIPTION OF THE DRAWINGS

FIG. 1 is a perspective view of a suspensory graft fixation device constructed in accordance with the principles of this invention situated in a bone tunnel.

FIG. 2 is a view of the suspensory graft fixation device of FIG. 1 showing a portion of the winding path of the suture loop.

FIG. 3 is a view of the suspensory graft fixation device of FIG. 1 showing a continuation of the winding path of suture.

FIG. 4 is a view of the suspensory graft fixation device of FIG. 1 showing a further continuation of the winding path of suture.

FIG. 5 is a diagrammatic representation of the suspensory graft fixation device of FIG. 1 showing its functional equivalency to a block and tackle pulley arrangement.

FIG. 6 is an alternate embodiment of the suspensory graft fixation device.

### DESCRIPTION OF THE INVENTION

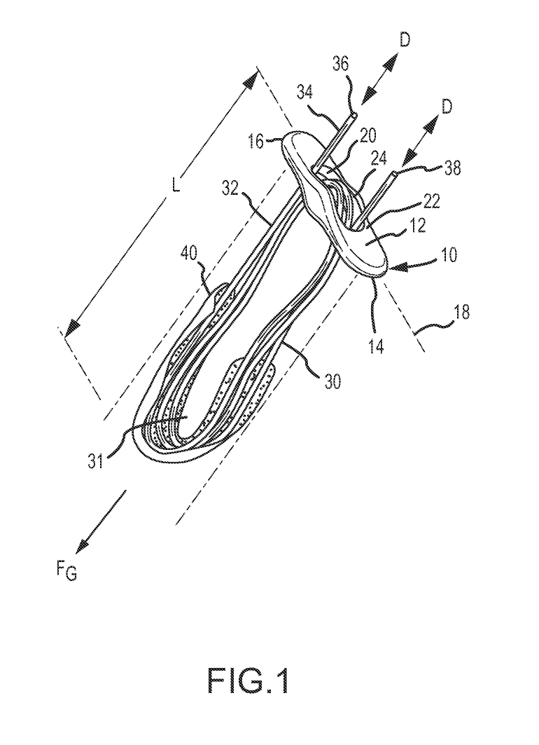

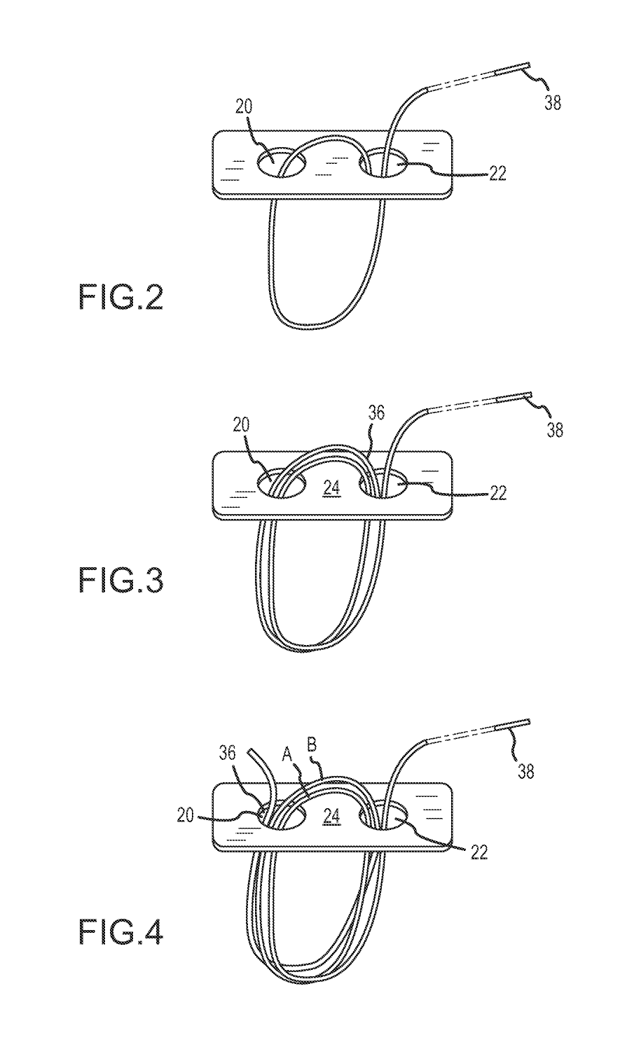

A suspensory graft fixation device 10 constructed in accordance with the principles of this invention is shown in FIG. 1. Device 10 comprises an elongated anchor member 12 having a first end 14, a second end 16, an axis 18, and a pair of suture receiving apertures 20 and 22. The side of the anchor member facing the bone surface will be sometimes herein referred to as the proximal side, while the opposite surface will be sometimes referred to herein as the distal side. Apertures 20 and 22 are situated on opposite sides of a central bridge portion 24 extending between them. Device 10 further comprises a graft supporting element in the form of a suture loop member 30 formed from a plurality of individual suture loops 32 which cooperate to form a graft ligament retaining loop 31 suspended from bridge 24. While bridge 24 in the preferred embodiment is the part of the anchor member between apertures 20 and 22, it will be understood that a transverse pin in the middle of a single opening (not shown) could form an equivalent pair of apertures. As shown in FIGS. 2 through 4, loop member 30 is formed from a single strand or length of suture or other filamentous material 34 wound in the same direction about a support element (i.e., bridge 24). Suture strand 34 has two ends 36 and 38 and loop member 30 is formed by repeatedly passing end 36 down through aperture 22 and up through aperture 20, and ultimately up out of aperture 20 on the last pass. The resulting loop member 30 has two distally extending free ends, each of which can move relative to the other, and in particular they can be pulled in a direction away from the graft retaining loop 31. The pulling direction D is in alignment along the length of the suture strand 34. The individual suture loops 32 may be made to pass through an optional slidable flexible cylindrical sleeve 40 situated at the proximal side of the device 10. This proximal portion of the loop is sometimes referred to as the sling or saddle 49. Anchor member 12 is slidably attached to one side of retaining loop 31 at the distal side of device 10. The terms proximal and distal are defined relative to the interior of the joint in which device 10 is used. Both the loops 32 and the sleeve 40 can be made from any flexible biocompatible material suitable for implantation. In the preferred embodiment they are made with high strength, filamentous material such as ultra high molecular weight polyethylene. In the preferred embodiment the anchor member is comprised of implantable grade Titanium. (In order to facilitate orienting elongated member 12 parallel to the bone tunnel axis and pulling it through the bone tunnel, an optional pulling-suture aperture (not shown) may be formed at the leading end of the member 12.)

Unique features of device 10 are its pulley-type characteristics, one of which is its ability to enable the adjustability of loop length 50 by simply pulling distally on one or both ends of suture 34. This is done by a system which operates in a way analogous to a plurality of pulleys. Not only does the system enable adjustable loop lengths, it also provides a mechanical advantage so that less force is needed to pull the graft into the tunnel than is used to hold the graft in place. This structure serves to shorten the loop length L while simultaneously presenting a large graft fixation force due to another pulley-type characteristic. By way of explanation, the theoretical mechanical advantage of the system shown here is the ratio of the force delivered by the system to the force put into it (ignoring friction). In the case of the suspensory device described herein the force delivered is the holding force F_{G} supporting the graft during use (the force exerted by the graft proximally on the interior side of loop member 30). That is, the holding force F_{G} pulling proximally on the saddle end of the suture loop. The force input into the system is F_{G}/4 (in the preferred embodiment with two complete loops and four strands passing between the ends of the loop member), the force required to pull distally to shorten the loop length L. The number of loops passing over bridge 24 is analogous to the number of pulleys. As a result of the pulley analogy there is a mechanical advantage created whereby the pulling force F_{G}/4 exerted on the suture ends 36, 38 at the distal end of the loop is multiplied at the proximal end of the loop where the graft ligament is situated.

In the preferred embodiment as best seen in FIGS. 2, 3 and 4 the pulley effect may be witnessed during use by the rotation of loops A and B around bridge 24 as the length L of loop member 30 is shortened by pulling suture ends 36 and 38 distally.

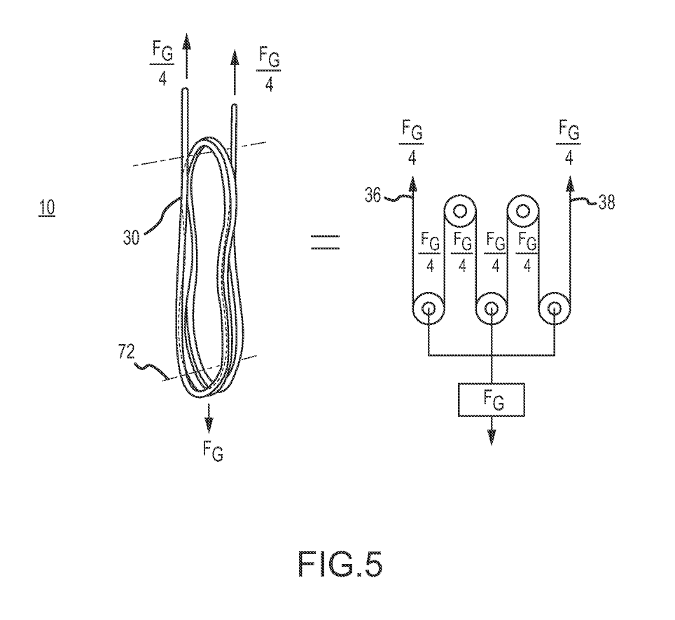

FIG. 5 is a diagrammatic representation of device 10 without the anchor member and the optional sleeve. Line 70 represents bridge 24 and line 72 represents saddle 49. Device 10 is shown to be equivalent to a block and tackle pulley arrangement.

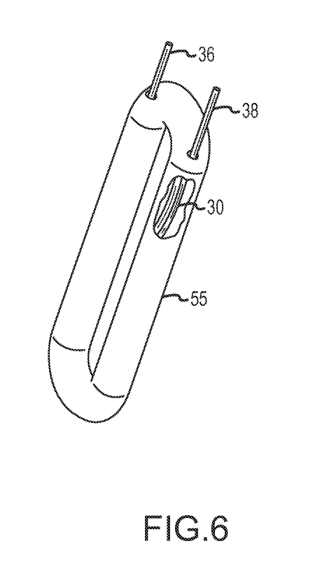

FIG. 6 is an alternate embodiment where anchor member 12 may be eliminated and a sleeve 55 may entirely encase the loop member 30 with small exit apertures for the suture tails 36 and 38.

An additional advantage of this invention is its ability to provide graft fixation in bone tunnels shorter than those required for prior art suspensory graft repairs. This invention is particularly useful for suspensory type repairs of the ACL or PCL in situations where the bone tunnel length is relatively short. For example, to perform an ACL surgery using a conventional transverse button on the lateral femoral surface, the surgeon must drill a trans-femoral tunnel comprising a proximal graft receiving portion and a smaller distal portion opening at the bone tunnel exit onto the lateral femoral cortex. The surgeon must then precisely measure the overall tunnel length and determine the graft tunnel-contact length desired as well as the loop length required to achieve the desired contact between the tunnel and the graft. The surgeon then calculates the drilling length of the graft tunnel socket by selecting the length of graft desired to remain in the tunnel and, in the case of a button with a continuous (i.e., endless) loop, adding the distance needed to deploy (i.e., flip) the button. The reason the added distance is necessary is because the button must completely exit the aperture prior to toggling perpendicular to the exit hole. A typical example of this would be an overall trans-femoral tunnel length of 45 mm where 20 mm of graft is desired to remain in the tunnel and 15 mm is needed to deploy the device. In this case the surgeon would drill the femoral socket 35 mm deep. Once the procedure is completed there will be excess space present between the femoral socket floor and the distal end of the graft. At this point, if one were using conventional techniques and devices the graft is in its final position. On the other hand, as will be understood below, use of the subject invention enables the advantageous elimination of this excess space and the maximization of graft contact with the bone tunnel wall where short trans-femoral tunnels are desired or encountered.

In particular, the method of ACL repair using the subject invention does not require extra tunnel length to allow the button to turn. The invention allows the loop to be sufficiently long so that the button may be passed through the femoral tunnel and deployed on the surface of the bone. The loop length can be made long enough to enable it to be accessible from inside the joint but outside the femoral tunnel so that the graft retaining loop 31 may be engaged by the graft ligament (folded about loop 31) even after the button has been flipped. The graft ligament, thus supported directly by the plurality of loops 32, is then pulled into the bone tunnel by pulling the suture ends 36 and 38 distally and with relatively little effort as loop length 50 is shortened. The graft may be pulled into the tunnel until it contacts the socket floor. The optional flexible sleeve 40 could be used to protect the graft from abrasion as well as to keep the individual loops 31 together. Once the graft is in the correct position an optional knot may be tied over the button to complete the repair to ensure fixation.

While anchor member 12 is shown as a single, unitary member with apertures 20 and 22 having unbroken perimeters, alternate structures could be utilized. For example, anchor 12 could have any shape and apertures 20, 22 could each have a passage through their perimeters. While the preferred embodiment described herein was formed with two complete turns of suture through apertures 20 and 22, it will be understood that any number of individual loops 31 could be used between zero and any greater number. If one chose not to have any loop at all, the device would have no mechanical advantage but would still function in a short tunnel situation (although the suture ends would clearly need to be tied to complete the procedure). If one chose too many loops, friction between the adjacent suture strands might limit the mechanical advantage.

It will be understood by those skilled in the art that numerous improvements and modifications may be made to the preferred embodiment of the invention disclosed herein without departing from the spirit and scope thereof.

What is claimed is:

1. A suspensory graft fixation device for securing a ligament graft in a bone tunnel comprising:
an elongated anchor member adapted to be situated transversely adjacent the exit of said bone tunnel;
a graft supporting element secured to said anchor member, said graft supporting element adapted to support said ligament graft at a predetermined variable distance away from said exit; and
pulley-type means for adjusting said distance.

2. A suspensory graft fixation device according to claim 1 wherein said graft supporting element comprises a plurality of loops formed from a single wound strand of filamentous material having two ends movable relative to each other.

3. A suspensory graft fixation device according to claim 2 further comprising a pair of apertures for receiving said plurality of loops, said apertures situated on opposite sides of the bridge portion of said anchor member.

4. A suspensory graft fixation device according to claim 1 wherein said pulley-type adjusting means comprises:
a plurality of loops formed from a single strand of filamentous material wound around the bridge of said anchor member such that the ends of said strand are movable relative to each other along the length of said strand and away from said anchor member.

5. A suspensory graft fixation device for securing a ligament graft in a bone tunnel comprising:
an elongated anchor member having a proximal side adapted for being situated transversely adjacent the exit of the bone tunnel, and a distal side, said suspensory anchor member having two apertures therethrough extending between said sides;
pulley-type graft support member interposed between said anchor member and the ligament graft.

6. A suspensory graft fixation device according to claim 5 wherein said apertures are spaced apart a predetermined distance on opposite sides of a support bridge and further comprising:
an elongated strand of filamentous material looped at least two complete times through said apertures to form a graft ligament support sling, the ends of said strand accessible from the distal side of said anchor member, whereby a graft ligament supported in said support sling will exert a first predetermined force directed proximally against the interior of said sling and whereby a second predetermined force applied distally to at least one of said strand ends will decrease the length of said graft support sling, said first predetermined force being a predetermined multiple of said second predetermined force.

7. A suspensory graft fixation device according to claim 6 wherein said filamentous material comprises suture.

8. A suspensory graft fixation device for securing a ligament graft in a bone tunnel comprising:
a plurality of loops of filamentous material formed by successively passing one end of said material proximally through said first aperture and then distally through said second aperture and ultimately out from said second aperture.

9. Method of adjusting the loop length of a suspensory graft fixation device comprising the steps of:
providing a suspensory graft fixation device according to claim 2; and
pulling at least one end of said filamentous material in order to shorten said loop length.
